(19) United States
(12) Patent Application Publication
(10) Pub. No.: US 2026/0057994 A1
(43) Pub. Date: Feb. 26, 2026

(54) INFUSION SITE FAILURE DETECTION

(71) Applicant: Eli Lilly and Company, Indianapolis, IN (US)

(72) Inventors: Eyal DASSAU, Acton, MA (US); Michelle Lynne KATZ, Newton, MA (US); Xinyue CHANG, Indianapolis, IN (US); Jie XUE, Zionsville, IN (US)

(21) Appl. No.: 19/104,909

(22) PCT Filed: Aug. 31, 2023
(86) PCT No.: PCT/US2023/073209

§ 371 Date: Feb. 19, 2025

Related U.S. Application Data
(60) Provisional application No. 63/374,443, filed on Sep. 2, 2022.

(51) Int. Cl. G16H 20/17; G16H 50/20

(52) U.S. Cl. CPC G16H 20/17; G16H 50/20

(57) ABSTRACT

Systems, methods, and devices are provided for predicting a status of an infusion site. Approaches include applying a regression model to physiological glucose data and insulin delivery data to generate predictive data, operating a trained machine learning model to process the predictive data to generate an output, and determining that the infusion site has failed or is likely to have failed based on the output.

### FIELD OF THE DISCLOSURE

The present disclosure relates to detecting issues with medication infusion sites.

### BACKGROUND

Subcutaneous insulin replacement therapy has proven to be the regimen of choice to control diabetes. Insulin is administered via either multiple daily injections or an infusion pump with dosages being informed by capillary glucose measurements made several times a day by a blood glucose meter. This conventional approach is known to be imperfect as day to day (and in fact moment to moment) variability can be significant. Further, this approach can be burdensome to the patient as it requires repeated finger sticks, a rigorous monitoring of food intake, and vigilant control of insulin delivery. The advent of glucose measurement devices such as a continuous glucose monitor creates the potential to develop insulin delivery systems such as closed loop systems that can automatically calculate and adjust insulin delivery amounts in response to measured glucose levels.

### SUMMARY

Insulin delivery systems may deliver the calculated insulin delivery amounts using an infusion set coupled to a patient's body at an infusion site. Over time, however, the site where the infusion set is positioned may become less effective and fail. Certain embodiments of the present disclosure are accordingly directed to systems, methods, and devices for detecting infusion site failure.

In Example 1, a method for predicting a status of an infusion site includes applying a regression model to physiological glucose data and insulin delivery data to generate predictive data. The method further includes operating a trained machine learning model to process the predictive data to generate an output. The method further includes determining that the infusion site has failed or is likely to have failed based on the output.

In Example 2, the method of Example 1, further including: generating an alert signal indicating that the infusion site has failed and displaying an alert on a graphical user interface in response to the alert signal.

In Example 3, the method of any of the preceding Examples, wherein the physiological glucose data and insulin delivery data is aggregated over a time period beginning when the infusion site was first in use.

In Example 4, the method of Example 3, further including: calculating a linear regression based on the physiological glucose data and insulin delivery data that is aggregated, wherein the predictive data is based, at least in part, on the linear regression.

In Example 5, the method of any of the preceding Examples, wherein the output is a value indicating a likelihood of infusion site failure.

In Example 6, the method of any of the preceding Examples, wherein the physiological glucose data and the insulin delivery data are updated and aggregated according to a set schedule.

In Example 7, the method of any of the preceding Examples, wherein predictive data comprises p values of selected metrics.

In Example 8, the method of Example 7, wherein the selected metrics include metrics selected from the categories of metrics, including physiological glucose variability and physiological glucose.

In Example 9, the method of Examples 7 or 8, wherein the selected metrics comprise: time above range, low glucose index, total bolus, standard deviation, coefficient of variation, and lability index.

In Example 10, the method of any of the preceding Examples, wherein the trained machine learning model is an XGBoost model.

In Example 11, the method of any of the preceding Examples, wherein the trained machine learning model is customized for a patient by retraining the machine learning model using prior physiological glucose data and insulin delivery data of the patient.

In Example 12, the method of any of the preceding Examples, further including: updating, on a user interface, a status icon associated with the infusion site based on the output.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a method for predicting a status of an infusion site includes applying—using an electronic controller—a regression model to physiological glucose data and insulin delivery data to generate predictive data. The method further includes operating a trained machine learning model—using the electronic controller—to process the predictive data to generate an output. The method further includes determining—by the electronic controller—that the infusion site has failed or is likely to have failed based on the output.

In Example 17, the method of Example 1, further including: generating an alert signal indicating that the infusion site has failed and displaying an alert on a graphical user interface in response to the alert signal.

In Example 18, the method of any of the preceding Examples, wherein the physiological glucose data and insulin delivery data is aggregated over a time period beginning when the infusion site was first in use.

In Example 19, the method of Example 3, further including: calculating a linear regression based on the physiological glucose data and insulin delivery data that is aggregated, wherein the predictive data is based, at least in part, on the linear regression.

In Example 20, a non-transitory computer-readable medium includes instructions that cause a hardware processor to: (1) apply a regression model to physiological glucose data and insulin delivery data to generate predictive data, (2) operate a trained machine learning model to process the predictive data to generate an output, and (3) determine that an infusion site has failed or is likely to have failed based on the output.

In Example 21, the non-transitory computer-readable medium of Example 20, wherein the output is a value indicating a likelihood of infusion site failure.

In Example 22, the non-transitory computer-readable medium of Example 20, wherein the selected metrics include metrics selected from the categories of metrics, including physiological glucose variability and physiological glucose.

In Example 23, the non-transitory computer-readable medium of Example 20, wherein the trained machine learning model is customized for a patient by retraining the machine learning model using prior physiological glucose data and insulin delivery data of the patient.

In Example 24, a system includes a controller including a processor and memory. The memory stores instructions that cause the processor to: apply a regression algorithm to physiological glucose data and insulin delivery data to generate predictive data, operate a trained machine learning model to process the predictive data to generate an output, and, based on the output, determine that an infusion site has failed or is likely to have failed.

In Example 25, the system of Example 24, wherein the output is a value indicating a likelihood of infusion site failure.

In Example 26, the system of Example 25, wherein the instructions further cause the processor to generate an alert signal indicating that the infusion site has failed when the likelihood is above a threshold. The system further includes: a user interface arranged to receive the alert signal and responsively generate an alert on the user interface.

In Example 27, the system of Example 24, wherein predictive data comprises p values of selected metrics.

In Example 28, the system of Example 27, wherein the selected metrics include metrics selected from categories of metrics, wherein the categories include physiological glucose variability and physiological glucose.

In Example 29, the system of Example 27, wherein one of the selected metrics is mean glucose.

In Example 30, the system of Example 24, wherein the physiological glucose data comprises summarized data based on a raw glucose data.

In Example 31, the system of Example 24, wherein the trained machine learning model is customized for a patient by retraining the machine learning model using prior physiological glucose data and prior insulin delivery data of the patient.

In Example 32, the system of Example 24, wherein the controller further includes a rule-based algorithm.

In Example 33, the system of Example 24, wherein the rule-based algorithm is invoked after a pre-determined period of time.

In Example 34, the system of Example 24, further including: a medication delivery device configured to deliver insulin to the patient and to generate the insulin delivery data.

In Example 35, the system of Example 34, further including: a glucose measurement device in communication with the controller and configured to generate the physiological glucose data.

In Example 36, a non-transitory computer-readable medium including instructions that cause a hardware processor to carry out the processes described herein.

### BRIEF DESCRIPTION OF THE DRAWINGS

FIG. 1 shows a schematic of a system for controlling physiological glucose, in accordance with certain embodiments of the present disclosure.

FIG. 2 shows an infusion set, in accordance with certain embodiments of the present disclosure.

FIG. 3 shows a block diagram of a method for detecting infusion site failure using a model-based approach, in accordance with certain embodiments of the present disclosure.

FIG. 4 shows graphs of raw and processed glucose data, in accordance with certain embodiments of the present disclosure.

FIG. 5 shows a block diagram of a method for detecting infusion site failure using a rule-based approach, in accordance with certain embodiments of the present disclosure.

FIGS. 6-8 show logic which may be used as part of the rule-based approach for detecting infusion site failure, in accordance with certain embodiments of the present disclosure.

FIG. 9 shows a graph showing regions for calculating area under the curve, in accordance with certain embodiments of the present disclosure.

FIG. 10 shows a block diagram of a method for detecting infusion site failure using both a model-based approach and a rule-based approach, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described but instead is intended to cover all modifications, equivalents, and alternatives falling within the scope the appended claims.

### DETAILED DESCRIPTION

Insulin delivery systems such as closed loop systems automatically calculate and adjust insulin delivery amounts in response to measured glucose levels. The systems deliver the calculated insulin delivery amounts using an infusion set coupled to a patient's body. Over time, the site where the infusion set is positioned may become less effective, resulting in a site failure. For example, the failure may be due to inflammation or other tissue degradation at the site that reduces the effectiveness of insulin delivered at the site. Other examples of failure may include site leakage, hyperglycemia and ketones in the blood, blood in the tube, etc. If the infusion site fails and is not addressed (e.g., by replacing the infusion set with a new set and/or using a different infusion site), patients may experience prolonged hyperglycemia. Certain embodiments of the present disclosure are accordingly directed to systems, methods, and devices for detecting infusion site failure.

The description below discloses various approaches for detecting infusion site failure. These approaches include a model-based approach, a rule-based approach, and a combined model-based and rule-based approach. Before detailing these approaches, the description herein outlines an example system in which these approaches may be incorporated. The approaches described herein may be utilized in other systems as well.

### System Hardware

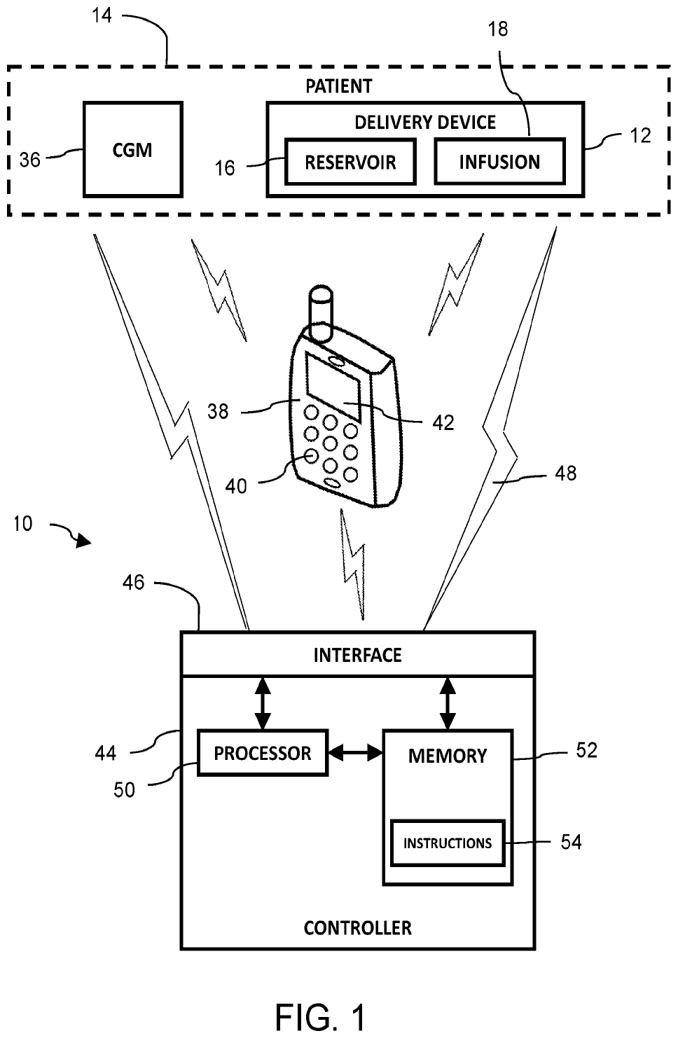

FIG. 1 depicts an exemplary representational block diagram of a system 10 for controlling physiological glucose. The system 10 includes a medication delivery device 12 such as an infusion pump which is removably coupled to a patient 14 via an infusion set 18. The medication delivery device 12 includes at least one medication reservoir 16 which contains a medication such as insulin, for example, although other suitable medications may be delivered with system 10. The medication delivery device 12 may deliver the medication to the patient 14 via infusion set 18, which provides a fluid path from the medication delivery device 12 to the patient 14. In other embodiments, the delivery device 12 may include an infusion catheter coupled directly to patient's subcutaneous tissue at the infusion site without use of an infusion set.

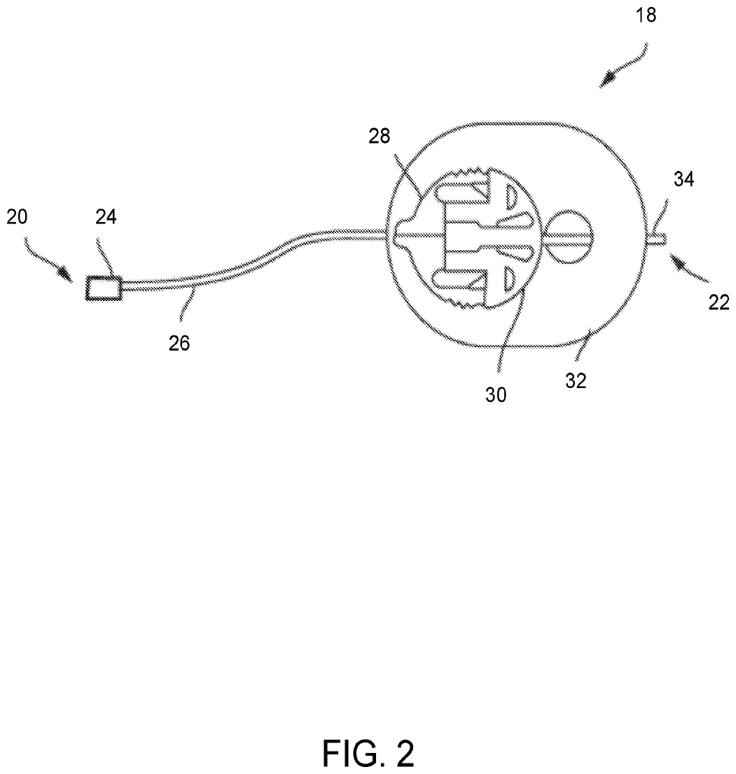

FIG. 2 shows an exemplary infusion set 18. The infusion set 18 includes a first, proximal end 20 that communicates with medication reservoir 16 (of FIG. 1) of an infusion pump to receive the medication and a second, distal end 22 that communicates with the patient 14 to deliver the medication. At the first end 20, the infusion set 18 includes a reservoir connector 24 configured to couple with the insulin reservoir, a flexible line set tubing 26, and a base connector 28 in the shape of a male buckle portion. At the second end 22, the infusion set 18 includes an infusion base 30 in the shape of a female buckle portion configured to receive the base connector 28, an adhesive pad 32 configured to adhere the infusion base 30 to the patient's skin, and an infusion catheter 34 (e.g., a needle or cannula) configured for insertion into the patient's skin. In use, the medication is directed from the medication delivery device 12, through the line set tubing 26, through the infusion catheter 34, and into the patient's subcutaneous tissue. The infusion set 18 of FIG. 2 is just one example of various types of infusion sets that may be used in the system 10.

Referring back to FIG. 1, the system 10 also includes an analyte sensor such as a glucose measurement device 36. The glucose measurement device 36 may be a standalone device or may be an ambulatory device. One example of a glucose measurement device is a continuous glucose monitor (CGM). In specific embodiments, the glucose measurement device 36 may be a glucose sensor such as a Dexcom G6 series continuous glucose monitor, although any suitable continuous glucose monitor may be used. The glucose measurement device 36 is illustratively worn by the patient 14 and includes one or more sensors in communication with or monitoring a physiological space (e.g., an interstitial or subcutaneous space) within the patient 14 and able to sense an analyte (e.g., glucose) concentration of the patient 14. In some embodiments, the glucose measurement device 36 reports a value that is associated with the concentration of glucose in the interstitial fluid, e.g., interstitial glucose. The glucose measurement device 36 may transmit a signal representative of an interstitial glucose value to the various other components of the system 10.

The system 10 includes a user interface device 38 (hereinafter the “UI 38”) that may be used to input user data to the system 10, modify values, and receive information, prompts, data, etc., generated by the system 10. In certain embodiments, the UI 38 is handheld user device programmed specifically for the system 10 or may be implemented via an application or app running on the medication delivery device 12 or a personal smart device such as a phone, tablet, watch, etc. The UI 38 may include input devices 40 (e.g., buttons, switches, icons) and a display 42 that displays a graphical user interface. The user may interact with the input devices 40 and the display 42 to provide information (e.g., alphanumeric data) to the system 10. In certain embodiments, the input devices 40 are icons (e.g., dynamic icons) on the display 42 (e.g., touchscreen). In one example, a patient uses the UI 38 to announce events such as a meal, start of exercise, end of exercise, emergency stop, etc.

The system 10 also includes an electronic controller 44. Although the controller 44 is shown as being separate from the medication delivery device 12 and the UI 38, the controller 44 may be physically incorporated into either the medication delivery device 12 or the UI 38 or carried out by a remote server. Alternatively, the UI 38 and the medication delivery device 12 may each include a controller 44 and control of the system 10 may be divided between the two controllers 44. For example, some functions and processes described herein may be carried out by a controller that is part of a remote server while other functions and processes are carried out by a controller that is part of the UI 38. Regardless of its physical location within the system 10, the controller 44 is shown as being directly or indirectly communicatively coupled to the medication delivery device 12, the glucose measurement device 36, and the UI 38.

The controller 44 may include or be communicatively coupled to one or more interfaces 46 to communicatively couple via one or more communication links 48 to the medication delivery device 12, the glucose measurement device 36, and/or the UI 38. Example interfaces 46 include wired and wireless signal transmitters and receivers. Example communication links 48 include a wired communication link (e.g., a serial communication), a wireless communication link such as, for example, a short-range radio link, such as Bluetooth, IEEE 802.11, a proprietary wireless protocol, and/or the like. The term “communication link” may refer to an ability to communicate some type of information in at least one direction between at least two devices. The communication links 48 may be a persistent communication link, an intermittent communication link, an ad-hoc communication link, and/or the like. Information (e.g., pump data, glucose data, drug delivery data, user data) may be transmitted via the communication links 48. The medication delivery device 12, the glucose measurement device 36, and/or the UI 38 may also include one or more interfaces to communicatively couple via one or more communication links 48 to the other devices in the system 10 such as a remote server.

The controller 44 illustratively includes at least one processor 50 (e.g., a microprocessor) that executes software (e.g., software modules) and/or firmware stored in memory 52 of the controller 44 and that is communicatively coupled to the one or more interfaces 46 and to each other. The software/firmware code contains instructions that, when executed by the processor 50, cause the controller 44 to perform the functions of the processes and functions described herein. The controller 44 may alternatively or additionally include one or more application-specific integrated circuits (ASICs), field-programmable gate arrays (FPGAs), digital signal processors (DSPs), hardwired logic, or combinations thereof. The memory 52 may include computer-readable storage media (e.g., a non-transitory computer-readable medium) in the form of volatile and/or nonvolatile memory and may be removable, non-removable, or a combination thereof. In embodiments, the memory 52 stores executable instructions 54 (e.g., computer code, machine-useable instructions, and the like) for causing the processor 50 to implement aspects of embodiments of system components discussed herein and/or to perform aspects of embodiments of methods and procedures discussed herein. The interfaces 46, the processor 50, and the memory 52 may be communicatively coupled by one or more busses. The memory 52 of the controller 44 is any suitable computer readable medium that is accessible by the processor. Memory 52 may be a single storage device or multiple storage devices, may be located internally or externally to the controller 44, and may include both volatile and non-volatile media. Exemplary memory includes random-access memory (RAM), read-only memory (ROM), electrically erasable programmable ROM (EEPROM), flash memory, CD-ROM, Digital Versatile Disk (DVD) or other optical disk storage, a magnetic storage device, or any other suitable medium which is configured to store data and which is accessible by the controller 44.

In the illustrated embodiment, the controller 44 receives information from a plurality of components of the system 10 and feed the information (e.g., pump data, glucose data, drug delivery data, user data) into a control algorithm which determines at least one drug delivery control parameter which may in part govern operation of the medication delivery device 12. In some specific embodiments, the controller 44 may receive pump data from the medication delivery device 12, glucose data from the glucose measurement device 36, and user data from the UI 38. The pump data received may include drug delivery data corresponding to drug dosages delivered to the patient 14 by the medication delivery device 12. The pump data may be supplied by the medication delivery device 12 as doses are delivered or on a predetermined schedule. The glucose data received by the controller 44 may include glucose concentration data from the glucose measurement device 36. The glucose data may be supplied at a continuous rate, occasionally or at predefined intervals (e.g., every 5 or 10 minutes).

The pump data, glucose data, drug delivery data, and user data may be provided to the controller 44 as acquired, on a predefined schedule or queued in the memory 52 and supplied to the controller 44 when requested. The user data may be input to the UI 38 in response to user/patient prompts generated by the UI 38 and/or declared by the patient 14 as instructed during training. In some embodiments, at least some of the pump data, glucose data, and/or user data may be retrieved from the memory 52 associated with the controller 44, and some of this data may be retrieved from a memory in the medication delivery device 12.

The at least one drug delivery parameter determined by the controller 44 may be a medication dose or doses, which may at least in part govern drug administration to the patient 14 via the medication delivery device 12. For insulin delivery (e.g., delivery of a rapid acting insulin or ultra-rapid acting insulin), the drug delivery parameter may be a basal rate (e.g., a basal profile including predefined time-varying insulin flow rates over the course of 24 hours), micro-bolus doses (e.g., corrected doses with respect to the basal rate), and/or a meal bolus. The basal delivery is the continuous delivery of insulin at the basal rate needed by the patient to maintain the glucose level in the patient's blood at the desired level outside of post-meal periods. Occasionally, the user may require a larger amount of insulin due to a change in activity such as eating a meal or other activities that affect the user's metabolism. This larger amount of insulin is herein referred to as a bolus. A meal bolus is a specific amount of insulin that is generally supplied over a short period of time. The nature of the medication delivery device 12 may require delivering the bolus as a continuous flow of insulin for a period or as a series of smaller, discrete insulin volumes supplied over a period. The meal bolus facilitates maintenance of the glucose level as the digestive system supplies a large amount of glucose to the blood stream.

The term physiological glucose herein refers to the measured concentration of glucose in the body. In some embodiments, physiological glucose may be the concentration of glucose in the blood, which may also be referred to as blood glucose. In other embodiments, physiological glucose may be the concentration of the glucose in the blood plasma, which may be referred to as plasma glucose. The measured value of plasma glucose is typically higher than blood glucose because the blood cells of the blood have been removed in the plasma glucose determination. The relationship between plasma glucose and blood glucose depends on the hematocrit and may vary from patient to patient and over time.

### Infusion Site Failure Detection

As noted above, an infusion site may become less effective and fail over time. The failure results in and is reflected as one or more of leakage, hyperglycemia and ketone, blood in the tube, inflammation at the infusion site, etc. However, removing an infusion set prematurely can increase the annual cost of the system. In addition to reducing annual cost, longer lasting infusion sets improve patient experience and the overall convenience of using the systems. Therefore, there is a desire and need for detecting infusion site failures.

The present disclosure describes different approaches for detecting infusion site failures. These approaches include a model-based approach, a rule-based approach, and a combined model-based and ruled-based approach. In certain embodiments, these approaches are used in near real-time while the patient is using an infusion pump and/or infusion set being analyzed. In such embodiments, the controller 44 and/or other processing device(s) may carry out one or more of these approaches and alert the patient and/or their medical provider when an infusion site has failed or is likely to have failed. The controller 44 may be part of the patient's smartphone and/or may be part of a remote server, which operates functions requiring more computing resources. In other embodiments, these approaches may be used after removal of the infusion set to help identify a root cause for hyperglycemia events. For example, instead of providing near real-time alerts, the approaches may be used to generate reports that classify root causes and severity levels of hyperglycemia events and whether they pertain to infusion site failure or other causes such as missed meal boluses or low meal boluses.

While the present disclosure refers to detecting infusion site failure in a system using an infusion set, the systems and methods herein may also be used to detect infusion site failures where an infusion catheter of a delivery device is coupled directly to the subcutaneous tissue of the patient without use of an infusion set.

### Model-Based Approaches

One approach for detecting infusion site failure is referred to herein as the model-based approach. In short, the model-based approach applies a trained machine learning model that outputs a prediction of whether a failure has occurred based on statistics derived from physiological glucose data and insulin delivery data. The model is operated such that predictions about the infusion site are generated periodically. In certain embodiments, the model is operated approximately once an hour, although other suitable periods or intervals may be implemented. While longer or shorter periods of time may be used, time periods such as one hour may reduce or minimize the power consumed by operating the model while also obtaining predictions in a period of time that may help prevent prolonged hyperglycemia. Once a likelihood of infusion site failure is predicted, an alert message may be sent to the patient and/or their medical provider.

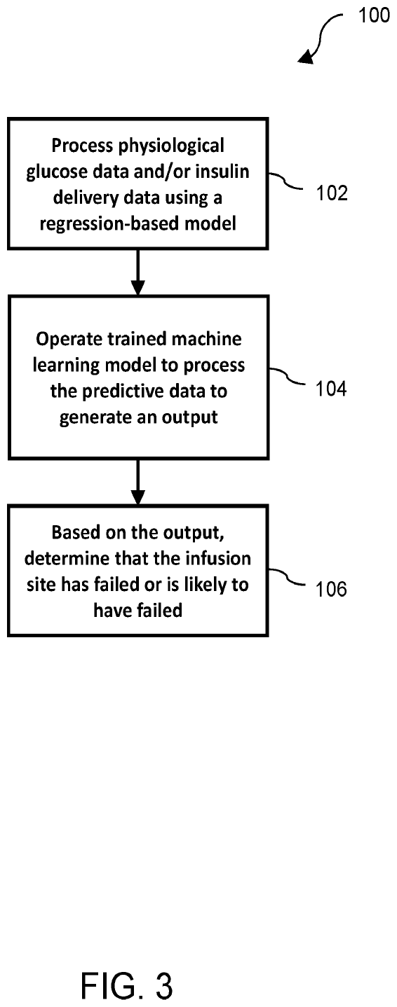

FIG. 3 outlines an exemplary method 100 for detecting infusion site failure using a model-based approach. Method 100 may be executed by processor 50 of controller 44, for example, based on pump data, glucose data, drug delivery data, and/or user data. In step 102, historical physiological glucose data and/or insulin delivery data of a patient is processed using a regression-based model or a regression algorithm. As noted above, physiological glucose data may be generated by and received from a sensor (e.g., a CGM coupled to the patient), and insulin delivery data may be generated by and received from a medical delivery device (e.g., a pump coupled to the patient). The physiological glucose data and/or insulin delivery data is aggregated and processed for each new time period beginning at when the infusion set was first in use and ending at the latest time when model is being operated. For example, if the patient has been wearing the infusion set for 2 days, then the historical physiological glucose data and/or insulin delivery data processed by the regression model would be 2 days of data. Then after one hour (when the model is run again), the past hour's data would be added to the last set of historical data and the updated set of historical data (e.g., two days and one hour's data) would be processed by the regression model.

The regression model outputs predictive data based on the aggregated physiological glucose data and insulin delivery data. In certain embodiments, processing the data using the regression algorithm includes calculating certain metrics (described herein) within consecutive one-hour time windows and then fitting a linear regression of the obtained sequence of metrics against time and calculating p values, which represent the significance of calculated time linear coefficients.

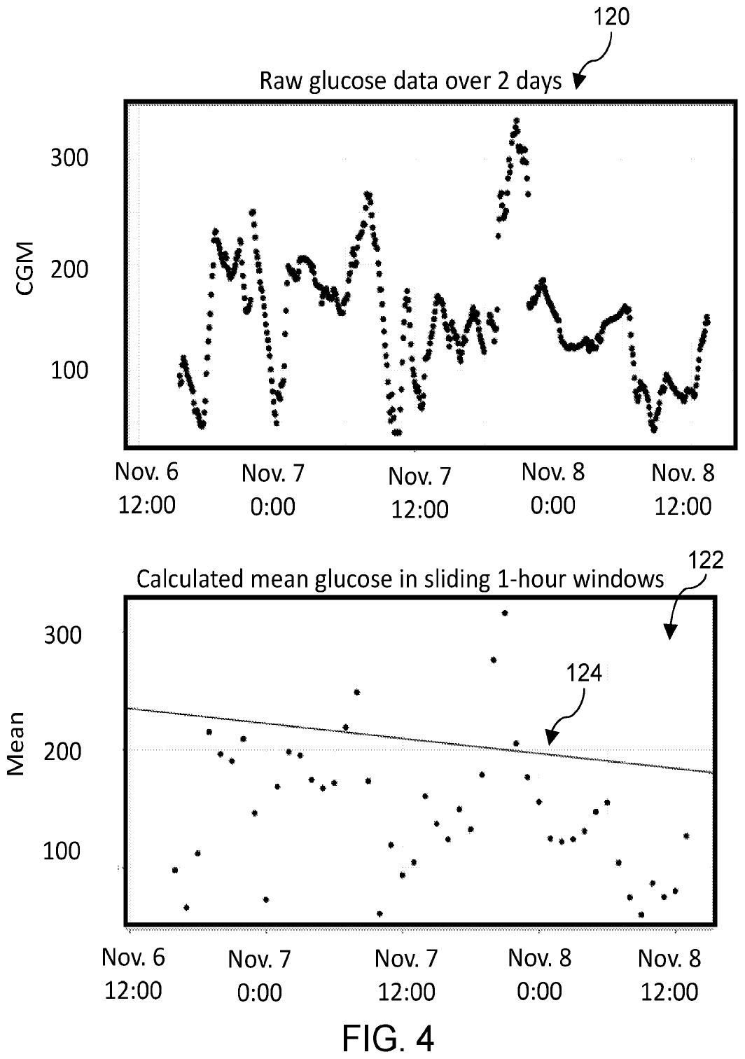

FIG. 4 provides an example of the above-described calculation of a metric and fitting of a linear regression. Graph 120 in FIG. 4 shows a plot of two days of raw glucose measurements (e.g., glucose levels measured every 5 or 10 minutes), and graph 122 shows calculated mean glucose within consecutive one-hour time windows. Graph 122 also includes a fitted linear regression 124 of the calculated mean glucose.

In certain embodiments, the predictive data comprises regression coefficient p values using selected metrics calculated at certain time intervals (e.g., 1-hour intervals). The selected metrics may comprise a combination of two or more of the following:

TABLE 1

| Category | Metrics |
|---|---|
| Glucose | Mean, Median |
| mean | |
| Glucose | SD (standard deviation), CV (coefficient of variation), |
| variability | J-index, Lability, GVP (glucose variability percentage), |
| | MAG (mean absolute glucose) |
| Glucose | TAR (time above rage), TBR (time below range), GRADE |
| risk | (glycemic risk assessment diabetes equation) for hyper, |
| | GRADE for hypo, M-value(hyper), M-value(hypo), LBGI |
| | (low blood glucose index), HBGI (high blood glucose |
| | index), ADRR (average daily risk range), PGS (post- |
| | challenge glucose spikes) |
| Insulin | Total bolus, AUC (area under curve) of IOB (insulin on |
| | board) |
| Others | Glucose change in last 7 hours, glucose slope from last |
| | 45 minutes, bolus slope from the beginning |

To reduce complexity and the computing resources required to operate the regression algorithm and the machine learning model (discussed further below), a subset of the metrics listed above may be selected. The subset of metrics may be selected based on how much independent influence a given metric has on the machine learning model. As one example, if two metrics have a high correlation (which suggests redundancy), then only one of the metrics may be selected. As another example, if a given metric has a low contribution to (or effect on) the output of the machine learning model, that metric will not be selected. As one specific example, if a given metric has a Shaply value of less than a predetermined amount (e.g., less than 0.25), then that metric is not selected.

Further, data other than that generated by the regression algorithm may be selected as features for input to the trained machine learning model. For example, the length of time the current infusion site has been used may be a selected feature.

In certain embodiments, the selected features—which are a combination of raw data and regression algorithm outputs—are those listed in the following table:

TABLE 2

| Features | Meaning |
|---|---|
| Length of wear | Number of days wearing infusion site |
| TAR p value | Linear coefficient p value in regression of time above |
| | range |
| LBGI p value | Linear coefficient p value in regression of low blood |
| | glucose index |
| Total bolus | Linear coefficient p value in regression of total bolus |
| p value | |
| SD p value | Linear coefficient p value in regression of standard |
| | deviation |
| CV p value | Linear coefficient p value in regression of coefficient of |
| | variation |
| Lability p value | Linear coefficient p value in regression of lability index |

The predictive data (e.g., p values) are generated by the regression algorithm. In step 104, the data (e.g., length of wear) and predictive data is inputted to a trained machine learning model to generate an output such as a prediction of infusion site failure.

The machine learning model is trained to receive the data and the predictive data as inputs, process the inputs, and then output a prediction of whether the infusion site has failed. The trained machine learning model may comprise one of several different types of models such as a neural network (e.g., deep learning model) and may apply one of several different types of algorithms (e.g., supervised algorithms such as random forest or logistic regression). In certain embodiments, the trained machine learning model is an XGBoost model.

In certain embodiments, the machine learning model for a given patient is updated over time as the model receives patient-specific data. For example, when the patient uses their first few infusion sets (e.g., first 3 months of infusion sets), the baseline machine learning model may be used. However, after the initial number of infusion sets or an initial period of time, the machine learning model may be adjusted to address patient-specific trends. For example, a machine learning model that is customized for a given patient may be created by (1) determining which sets of training data (e.g., 50-150 sets of training data) are most similar (e.g., using mean physiological glucose) to the patient's actual data over time and (2) retrain the machine learning model based on the determined sets of training data and the patient's actual data collected during the initial period of time. Additionally or alternatively, before the patient's actual data is used to retrain the machine learning model, the machine learning model must detect multiple infusion site failures such that the actual data has examples of infusion site failures and the data surrounding such detected failures.

In certain embodiments, the output of the trained machine learning model is a likelihood (e.g., a value on a scale of 0-100%) that the infusion site has failed (step 106). The output may be used in various ways to alert the patient and/or their medical provider. As one example, if the likelihood is above a certain value (e.g., 75%), then an alert signal is generated and sent to the user and/or their medical provider. The alert signal may cause an alert to be displayed on the UI 38. As another example, the likelihood may be used to periodically update a window or status icon displayed on the UI 38. In this example, the window or status icon displays the current status (e.g., within the past hour or since the last output) of the infusion set. This involves displaying the numerical value of the likelihood on the UI 38, displaying a battery-indicator-like graphic representing a remaining capacity of the infusion set, displaying a traffic light graphic that is responsive to the value of the likelihood, or simply displaying an icon in different colors that indicate the value of the likelihood. If the likelihood is below 50%, for example, then the window or status icon indicates that the infusion set is operating properly (e.g., by displaying a certain color, phrase, text, number, or icon). If the likelihood is 50% to 75%, then the window or status icon indicates that the infusion set may need to be replaced soon. And if the likelihood is greater than 75%, the window or status icon indicates that the infusion set needs to be changed and a separate alert is generated and displayed (e.g., in a pop-up window). The various numerical ranges discussed immediately above may be modified as needed.

The above-described model-based approach may be carried out on a server (e.g., in the cloud), on the UI 38 (e.g., via an application on a patient's smart phone), on the medication delivery device itself (e.g., via an insulin pump), or by a combination of system components.

### Rule-Based Approaches

The second disclosed approach for detecting infusion site failure is referred to in this description as the rule-based approach. In short, the rule-based approach applies a series of rules designed to determine whether blood glucose data indicating hyperglycemia is caused by an infusion site failure or another cause such as a missed meal bolus or a low meal bolus.

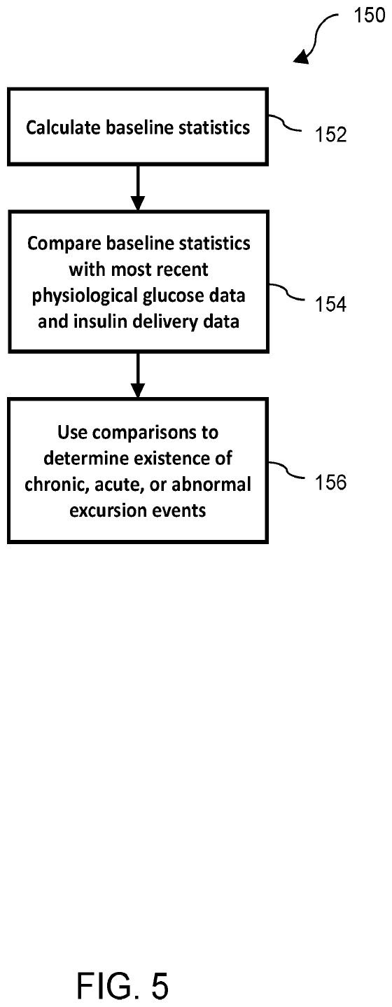

FIG. 5 outlines a method 150 for detecting infusion site failure using a rule-based approach. In step 152, baseline patient statistics are calculated. These baseline statistics may be based on a patient's physiological glucose data and insulin delivery data. For example, the baseline statistics may include mean physiological glucose, TAR (time above range), and nadir glucose and peak glucose during a baseline period. In certain embodiments, if a new infusion set has been worn for fewer than 3 days, the baseline period is initially the first 2 days. The baseline period may then be extended to the first 3 days if the new infusion set has been worn for 3 days or more. In certain embodiments, the method 150 is initiated only after the patient has worn a new infusion set for at least 2 days.

In step 154, the baseline statistics are compared to a patient's most recent physiological glucose data and insulin delivery data. In certain embodiments, the most recent data is data collected within the last 36 hours, even if this time period overlaps with the baseline time period.

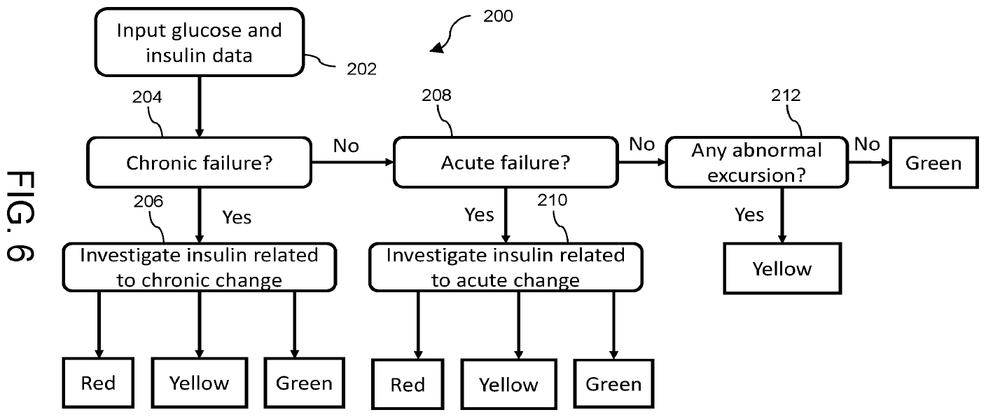

In step 156, in certain embodiments, the baseline statistics are (1) first compared to thresholds set to detect whether a chronic infusion site failure occurred, (2) then compared to thresholds set to detect whether an acute infusion site failure occurred, and (3) then compared to thresholds set to detect whether an abnormal excursion occurred. The various thresholds—described in further detail herein with respect to FIGS. 6-8—are set to detect a risk of hyperglycemia.

FIGS. 6-8 show additional details of detecting infusion site failure using the rule-based approach, as outlined in FIG. 5. The method 200 of FIG. 6 includes inputting physiological glucose data and insulin delivery data a computing device such as the controller 44 (step 202). The data is analyzed first to determine whether a chronic infusion site failure may have occurred at step 204. Steps 202 and 204 of the method 200 of FIG. 6 correspond to steps 152 and 154 of the method 150 of FIG. 5. The remaining description in this section provides further examples of step 156 of the method 150 of FIG. 5.

If the data suggests a chronic infusion site failure, the data is further investigated to distinguish between an infusion site failure or another cause of a chronic hyperglycemia event (step 206). Based on certain criteria described further below, logic dictates that a chronic infusion set failure has occurred (labeled as “Red” in FIG. 6), is likely to occur (labeled as “Yellow” in FIG. 6), or should be ignored (labeled as “Green” in FIG. 6). If the data does not suggest a chronic site failure, the data is analyzed secondly to determine whether an acute infusion site failure may have occurred at step 208. If the data suggests an acute infusion site failure, the data is further investigated to distinguish between an infusion site failure or another cause of an acute hyperglycemia event (step 210). Based on certain criteria described further below, logic dictates that an acute infusion set failure has occurred, is likely to occur, or should be ignored. Finally, if the data does not suggest an acute site failure, the data is analyzed to determine whether an abnormal excursion relating to an infusion site failure may have occurred at step 212. Logic may dictate that an infusion set failure has likely occurred or that the excursion should be ignored.

Chronic infusion site failures involve situations where the insulin delivery has gradually become less effective over time at the infusion site. As such, the thresholds for this first comparison to the baseline statistics are set to detect whether physiological glucose has increased to an extent that suggests a risk of hyperglycemia. For chronic infusion site failure, step 204 involves determining whether (1) the patient's TAR is higher than a threshold (e.g., 25%) and (2) has increased more than a threshold (e.g., 30%) from the baseline TAR. Step 204 may also involve determining whether (3) either nadir physiological glucose has increased more than a threshold (e.g., 70%) compared to the baseline statistics or mean physiological glucose has increased more than a threshold (e.g., 30%) from the baseline statistics. When these various thresholds have been breached, it suggests that there is a risk of hyperglycemia. However, additional criteria may be applied to determine whether the risk of hyperglycemia is due to a temporary increase in physiological glucose or a chronic infusion site failure.

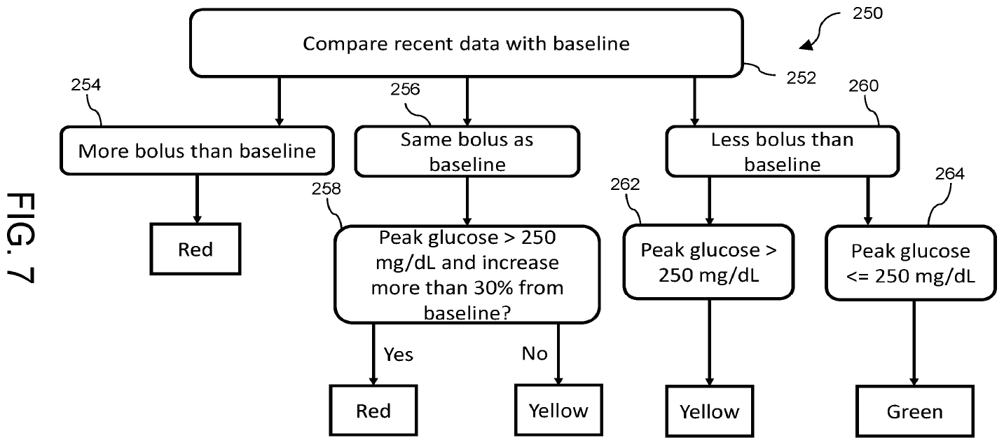

FIG. 7 outlines logic 250 for distinguishing between a temporary increase in physiological glucose or a chronic issue. At 252, the logic involves comparing the insulin delivery amounts from the baseline time period and the most recent time period. In certain embodiments, this comparison is carried out using the Wilcoxon rank-sum test. At 254, if the comparison shows that the most recent time period involves a greater amount of delivered insulin, then the logic dictates that a chronic insulin set failure is detected.

At 256, if the comparison shows that the most recent time period involves a similar amount of delivered insulin, then a second comparison is made at 258. This comparison involves determining whether (1) the peak physiological glucose of the most recent time period is above a threshold (e.g., 250 mg/dL) and (2) the peak physiological glucose of the most recent time period has increased by a threshold (e.g., 30%) compared to the baseline statistics. If both criteria of 258 are met, then the logic dictates that a chronic insulin set failure is detected. If not, the logic still dictates that there is some risk (but a lesser risk) of hyperglycemia chronic infusion site failure.

At 260, if the comparison shows that the most recent time period involves a smaller amount of delivered insulin, then a second comparison is made at 262. This comparison involves determining whether the peak physiological glucose of the most recent time period is above or below a threshold (e.g., 250 mg/dL). If the peak physiological glucose is above the threshold, the logic dictates that there is some risk (but a lesser risk) of hyperglycemia due to a chronic infusion site failure.

As noted above, after the baseline statistics are compared to thresholds set to detect a potential chronic infusion site failure (as described in the paragraphs immediately above), the baseline statistics are compared to thresholds set to detect a potential acute infusion site failure. Acute infusion site failure involves situations where the infusion site failure causes a relatively dramatic increase in physiological glucose. For acute infusion site failure, step 208 (of FIG. 6) involves determining whether physiological glucose has increased more than a threshold (e.g., 180 mg/dL) starting from a lower threshold (e.g., 70 mg/dL) in a certain time period (e.g., 7 hours). When such criteria have been met, it suggests that there is a risk of hyperglycemia. However, additional criteria may be applied to determine whether the risk of hyperglycemia is due to a reason other than an acute insulin site failure.

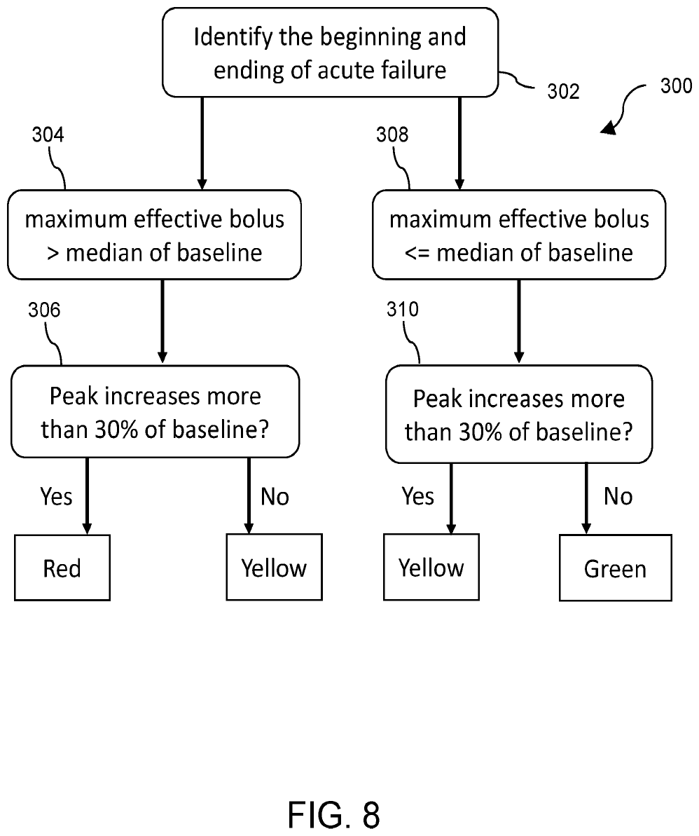

FIG. 8 outlines logic 300 for determining whether a dramatic increase in physiological glucose is a result of an acute insulin site failure or another reason. In short, if recent insulin boluses are greater than those delivered during the baseline time period, the acute increase in physiological glucose is likely due to an infusion site failure rather than a missed bolus or a low meal bolus. In certain embodiments, for acute failure analysis, the baseline time period is shorter (e.g., 12 hours) than the baseline time period for chronic failure analysis.

At 302, the logic involves determining the beginning time and the end time of the determined acute increase in physiological glucose. At 304, an amount of a past maximum effective bolus is determined. In certain embodiments, the past maximum effective bolus is calculated as the largest bolus taken over a set amount of time (e.g., 6 hours) prior to the beginning time and/or the end time of the determined acute increase in physiological glucose. Also, at 304, the past maximum effective bolus is compared to a median bolus from the baseline period. At 306, a peak physiological glucose of the most recent time period is compared to that of the baseline statistics. If the comparison shows that most recent peak is greater than a threshold (e.g., 30%) compared to the baseline, then the logic dictates that an acute insulin set failure is detected. If not, the logic still dictates that there is some risk (but a lesser risk) of hyperglycemia due to an acute infusion site failure. At 308, a peak physiological glucose of the most recent time period is compared to that of the baseline statistics. If the comparison shows that most recent peak is greater than a threshold (e.g., 30%) compared to the baseline, then the logic dictates that there is some risk (but a lesser risk) of hyperglycemia due to an acute infusion site failure.

As noted above, after the chronic failure analysis and the acute failure analysis, the rule-based approach may detect an abnormal excursion. An excursion involves situations where physiological glucose increases from a nadir to a peak above a threshold (e.g., 180 mg/dL) and then decreases below the threshold until a nadir appears. An excursion is abnormal if the physiological glucose and insulin delivery data indicate a declined capability of control, such as if physiological glucose does not quickly decrease after an insulin bolus is delivered. In an illustrative embodiment, detecting an abnormal excursion follows a multi-step process: (1) identify an excursion period, (2) calculate area under the curve (AUC) of hyperglycemia regions, and (3) analyze relevant insulin delivery data.

For the first step, an excursion period is determined by identifying two nadirs and one peak in the physiological glucose data (e.g., the time period starting with an increase in physiological glucose, a peak, and ending when physiological glucose stops decreasing). In certain embodiments, to help limit the effect of noise and/or minor local excursions, the physiological glucose data is processed (e.g., via a local polynomial method) to smooth out the physiological glucose data. And one excursion is considered not finished and combined with the next one under either of the two circumstances: (1) the ending nadir is more than 180 mg/dL, (2) ending nadir is between 70 mg/dL-180 mg/dL and glucose stay below 180 mg/dL for more than 2 hours from the current peak until the next peak.

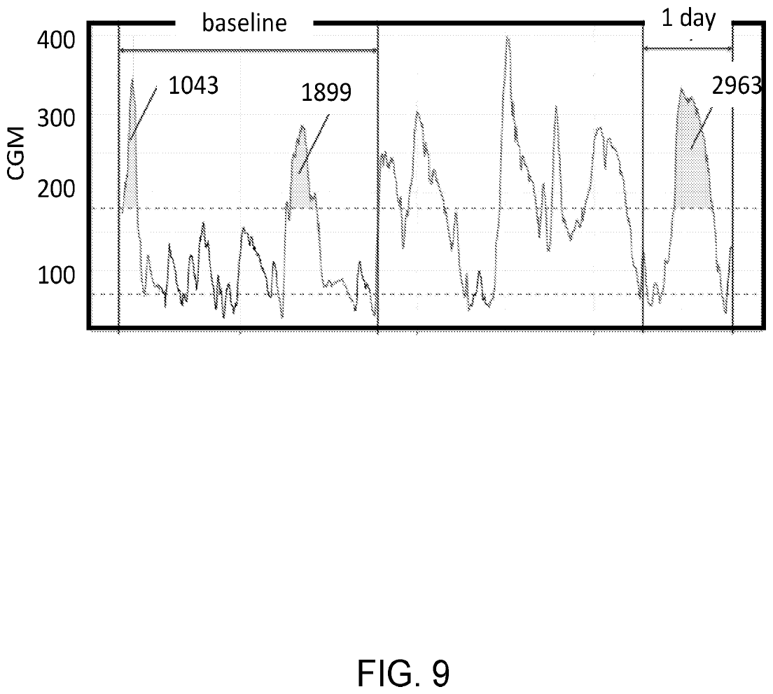

For each excursion period, there will be a region that is above a certain threshold (e.g., 180 mg/dL) and that may be referred to as the hyperglycemia region. The AUC for the hyperglycemia regions may be calculated for both the baseline time period and the most recent time period (e.g., the last 1 day). FIG. 9 shows an example plot of physiological glucose data and regions that define the AUC for hyperglycemia regions. The AUC is shown in FIG. 8 as shaded area with numerical indicators noting the calculated area. If any AUC of a hyperglycemia region during the most recent time period is larger than all the AUC in the baseline period, then the logic dictates further investigation.

The further investigation may involve determining indications of weakened physiological glucose responses to insulin deliveries. In certain embodiments, such indications are measured using a metric—referred to herein as insulin effect per unit—that measures the capability of reducing physiological glucose with insulin deliveries:

$$insulin effect per unit = \frac{glucose reduction}{{\int }_{𝒯} iob ( t ) dt}$$

where iob(t) is an estimated cumulative insulin on board at time t.

If the insulin effect per unit during any excursion is statistically lower than baseline, then insulin is having a less effective ability to reduce physiological glucose. In certain embodiments, a threshold is set from a baseline distribution created by bootstrap sampling approach to dictate whether the abnormal excursions are a result of an infusion site failure.

### Combined Model-Based and Rule-Based Approach

In certain embodiments, both a model-based approach and a rule-based approach are utilized in various ways to complement each other.

As one example, the two approaches are performed simultaneously to provide a check on the other approach. For instance, both approaches may be carried out periodically (e.g., once approximately every hour) and the results can be compared. If the rule-based approach determines an event has occurred (such as the “Red” or “Yellow” events shown in the Figures and described above), then the output of the model-based approach may be used to confirm or change the determined event. If the model-based approach outputs a prediction with a high-level of confidence that contradicts the output of the rule-based approach, the output of the model-based approach may be ultimately used to determine the type of event. For example, if the rule-based approach results in a Red event or a Yellow event, the event is changed to a Green event (e.g., no infusion site failure) if the probability of an event is less than a threshold (e.g., 5%). Conversely, if the rule-based approach Green event, the event is changed to a Red or Yellow (e.g., indicating an infusion site failure) if the probability of an event is greater than a threshold (e.g., 95%)

As another example, the rule-based approach may be solely used until an event has been detected. Once an event is detected, the model-based approach may be used to check the output of the rule-based approach. Using this combination of approaches may reduce the overall power consumption of the system because the rule-based approach requires fewer computing resources compared to the model-based approach.

As another example, the model-based approach may be used exclusively for an initial period of time (e.g., first 2 or 3 days) of wear because it is more sensitive with shorter period of data. After expiration of the initial period of time, the system may switch to the rule-based approach which is more stable with longer periods of data until the infusion set is removed.

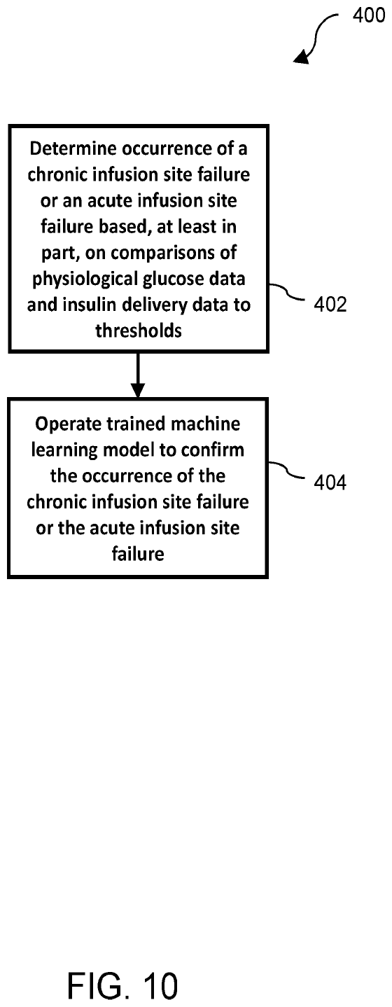

FIG. 10 illustrates a method 400 for using both a model-based approach and a rule-based approach. The method 400 includes determining occurrence of a chronic infusion site failure or an acute infusion site failure based, at least in part, on comparisons of physiological glucose data and insulin delivery data to thresholds (step 402). In certain embodiments, the comparisons are between thresholds and calculated differences between baseline physiological glucose data and insulin delivery data and recent physiological glucose data and insulin delivery data.

In response to determining occurrence of a chronic infusion site failure or an acute infusion site failure, a trained machine learning model may be operated to confirm the occurrence of the chronic infusion site failure or the acute infusion site failure (step 404). For example, the trained machine learning model can output a value indicating a likelihood of an infusion site failure. If the value is above and/or below a threshold, the occurrence of the chronic infusion site failure or the acute infusion site failure may be confirmed and an alert may be generated. In certain embodiments, the same physiological glucose data and insulin delivery data (or data derived therefrom) are used by both the rule-based approach and the model-based approach.

### Further Aspects of the Disclosure

In certain aspects, as noted above, a method utilizing rule-based approach can be applied for determining a status of an infusion site. The method includes calculating baseline statistics associated with an initial time period and the infusion site, determining that a difference between the baseline statistics and physiological glucose data from a later time period have crossed a first threshold, and determining occurrence of a chronic infusion site failure or an acute infusion site failure—in response to determining that the difference between the baseline statistics and the physiological glucose data has crossed the threshold.

Further aspects of the method include comparing a difference between the baseline statistics and insulin delivery data and determining that the physiological glucose data is above a second threshold.

In an additional aspect, the method includes determining that an aspect of insulin delivery data is higher than the baseline statistics and, in response, determining the occurrence of the chronic infusion site failure.

In an additional aspect, the method includes determining that an aspect of insulin delivery data is higher than the baseline statistics and, in response, determining the occurrence of the acute infusion site failure.

In other aspects, as noted above, a method can be applied for determining a status of an infusion site by combining one or more rule-based approaches described herein and one or more model-based approaches described herein. The method includes—using a rule-based approach—determining occurrence of a chronic infusion site failure or an acute infusion site failure based, at least in part, on comparisons of physiological glucose data and insulin delivery data to thresholds. The method further includes—using a model-based approach—operating a trained machine learning model to confirm the occurrence of the chronic infusion site failure or the acute infusion site failure, in response to determining occurrence of a chronic infusion site failure or an acute infusion site failure.

In an additional aspect, the rule-based approach is carried out more frequently than the model-based approach. For example, the rule-based approach may be used approximately once an hour, and the model-based approach may be used only after the rule-based approach determines an occurrence of a chronic infusion site failure or an acute infusion site failure.

In an additional aspect, the rule-based approach and the model-based approach are performed simultaneously.

In an additional aspect, an output of the model-based approach confirms or overrules the output of the rule-based approach. For example, if the output of the model-based approach indicates a high probability (e.g., 90% or higher, 95% or higher) that a chronic infusion site failure or an acute infusion site failure, the output of the model-based approach will overrule the output of the rule-based approach.

In an additional aspect, the same physiological glucose data and insulin delivery data (or the same data derived therefrom) are used by both the rule-based approach and the model-based approach.

Accordingly, the present disclosure is intended to embrace all such alternatives, modifications and variances. Additionally, while several embodiments of the present disclosure have been illustrated in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

1-15. (canceled)

16. A method for predicting a status of an infusion site, the method comprising:
applying, by an electronic controller, a regression model to physiological glucose data and insulin delivery data to generate predictive data;
operating, by the electronic controller, a trained machine learning model to process the predictive data to generate an output; and
based on the output, determining that the infusion site has failed or is likely to have failed.

17. The method of claim 16, further comprising:
generating an alert signal indicating that the infusion site has failed; and
displaying an alert on a graphical user interface in response to the alert signal.

18. The method of claim 16, wherein the physiological glucose data and insulin delivery data is aggregated over a time period beginning when the infusion site was first in use.

19. The method of claim 18, further comprising:
calculating a linear regression based on the physiological glucose data and insulin delivery data that is aggregated, wherein the predictive data is based, at least in part, on the linear regression.

20. A non-transitory computer-readable medium including instructions that cause a hardware processor to:
apply a regression model to physiological glucose data and insulin delivery data to generate predictive data;
operate a trained machine learning model to process the predictive data to generate an output; and
based on the output, determine that an infusion site has failed or is likely to have failed.

21. The non-transitory computer-readable medium of claim 20, wherein the output is a value indicating a likelihood of infusion site failure.

22. The non-transitory computer-readable medium of claim 20, wherein the selected metrics include metrics selected from the categories of metrics, including physiological glucose variability and physiological glucose.

23. The non-transitory computer-readable medium of claim 20, wherein the trained machine learning model is customized for a patient by retraining the machine learning model using prior physiological glucose data and insulin delivery data of the patient.

24. A system comprising:
a controller including a processor and memory, the memory storing instructions that cause the processor to:
apply a regression algorithm to physiological glucose data and insulin delivery data to generate predictive data,
operate a trained machine learning model to process the predictive data to generate an output, and
based on the output, determine that an infusion site has failed or is likely to have failed.

25. The system of claim 24, wherein the output is a value indicating a likelihood of infusion site failure.

26. The system of claim 25, wherein the instructions further cause the processor to generate an alert signal indicating that the infusion site has failed when the likelihood is above a threshold, the system further comprising:
a user interface arranged to receive the alert signal and responsively generate an alert on the user interface.

27. The system of claim 24, wherein the predictive data comprises p values of selected metrics.

28. The system of claim 27, wherein the selected metrics include metrics selected from categories of metrics, wherein the categories include physiological glucose variability and physiological glucose.

29. The system of claim 27, wherein one of the selected metrics is mean glucose.

30. The system of claim 24, wherein the physiological glucose data comprises summarized data based on a raw glucose data.

31. The system of claim 24, wherein the trained machine learning model is customized for a patient by retraining the machine learning model using prior physiological glucose data and prior insulin delivery data of the patient.

32. The system of claim 24, wherein the controller further includes a rule-based algorithm.

33. The system of claim 24, wherein the rule-based algorithm is invoked after a pre-determined period of time.

34. The system of claim 24, further comprising:
a medication delivery device configured to deliver insulin to the patient and generate the insulin delivery data.

35. The system of claim 34, further comprising:
a glucose measurement device in communication with the controller and configured to generate the physiological glucose data.
